(19) United States
(12) Patent Application Publication
(10) Pub. No.: US 2012/0178948 A1
(43) Pub. Date: Jul. 12, 2012

(54) PROCESS FOR THE HYDROGENATION OF 1,4-BUTYNEDIOL TO TETRAHYDROFURAN IN THE GAS PHASE

(75) Inventors: Rolf PINKOS, Bad Duerkheim (DE); Olga Osetska, Mannheim (DE); Lucia Königsmann, Stuttgart (DE)

(73) Assignee: BASF SE, Ludwigshafen (DE)

(21) Appl. No.: 13/346,144

(22) Filed: Jan. 9, 2012

Related U.S. Application Data
(60) Provisional application No. 61/431,868, filed on Jan. 12, 2011.

(51) Int. Cl. C07D 307/08

(52) U.S. Cl. 549/509

(57) ABSTRACT

The present invention relates to a process for the catalytic hydrogenation of 1,4-butynediol to tetrahydrofuran at at least the decomposition temperature of 1,4-butynediol, wherein 1,4-butynediol is vaporized in a hydrogen-comprising gas stream and is hydrogenated in gaseous form over at least one catalyst, comprising at least one of the elements from groups 7 to 11 of the Periodic Table of the Elements.

The present application incorporates the prior U.S. application 61/431868 submitted on Jan. 12, 2011, by reference.

The process of the invention relates to the catalytic hydrogenation of 1,4-butynediol (BYD) to tetrahydrofuran (THF) in the gas phase over heterogeneous catalysts in the presence of hydrogen.

THF is widely used as a solvent and serves as starting material for polytetrahydrofuran. It is produced in an amount of several thousand metric tons every year worldwide. Various possible ways of obtaining it are known. Thus, maleic anhydride can be prepared from butane and can then, for example as diester, be hydrogenated to THF, GBL (gamma-butyro-lactone), BDO (1,4-butanediol) (K. Weissermel, H.-J. Arpe, Industrielle Organische Chemie, 5th edition 1998, page 408). A further customary route to THF is the acid-catalyzed cyclization of 1,4-butanediol (K. Weissermel, H.-J. Arpe, Industrielle Organische Chemie, 5th edition 1998, page 113).

The technology for producing THF which is most widespread industrially over the world is cyclization of 1,4-butanediol which is in turn usually prepared by hydrogenation of 1,4-butynediol, for example as described in DE-A 19641707, usually under a considerable pressure in the range from 2 to 30 MPa. Here, it has to be noted in the reaction of butynediol that the latter can decompose under thermal stress, especially in the presence of impurities, in particular metals and salts thereof. In the case of the pure material, this generally occurs at and above 160° C., in particular above 200° C. Impurities, in particular heavy metals, can decrease this decomposition temperature further.

It would be economically desirable, because of lower capital costs, for the hydrogenation of 1,4-butynediol to give THF directly without 1,4-butanediol having to be isolated as intermediate.

Processes for the direct preparation of THF from butynediol have been described exclusively in the liquid phase. These are at present not carried out industrially. DE-A 2029557 describes a process for the direct preparation of THF from 1,4-butynediol in the liquid phase. According to the disclosure of DE-A 2029557, butynediol is converted into THF and water in the liquid phase in the presence of a solvent over a catalyst having a hydrogenation and acid function. Reworking the examples indicates that the process cannot be carried out. Mainly butanediol is found as product, but only negligible amounts of THF.

It was therefore an object of the present invention to provide a process for the catalytic hydrogenation of 1,4-butynediol to THF, which displays good selectivities and high catalyst operating lives and provides a direct route to THF from 1,4-butynediol which is industrially interesting because of its economics.

It has now surprisingly been found that THF can be obtained in high yield by catalytic hydrogenation of 1,4-butynediol at at least the decomposition temperature of 1,4-butynediol by vaporizing 1,4-butynediol in a hydrogen-comprising gas stream and hydrogenating it in gaseous form over at least one catalyst comprising at least one of the elements of groups 7 to 11 of the Periodic Table.

The temperature for vaporization and the hydrogenation temperature are in the range 160-330° C., preferably 160-300° C., particularly preferably 170-280° C. The temperature at which the 1,4-butynediol is vaporized can, within the abovementioned temperature range, be lower than the hydrogenation temperature. The pressure during vaporization and hydrogenation is from 0.05 MPa (megapascal) to 10 MPa absolute, preferably from 0.1 to 6 MPa absolute, particularly preferably from 0.15 to 2 MPa absolute. The pressure during vaporization corresponds to at least the pressure of the hydrogenation.

1,4-Butynediol can be used as pure material, but preference is given to using 1,4-butynediol as is obtained from the synthesis of 1,4-butynediol. This technical-grade butynediol comprises, for example, water, propynol, formaldehyde in free form or bound as hemiacetals or acetals, methanol and also small amounts, generally less than 1%, of acetylene, dissolved or solid materials such as catalyst constituents from the 1,4-butynediol synthesis catalyst (e.g. copper salts such as copper acetylides which increase the susceptibility of butynediol to decompose) or salts originating from pH regulation, e.g. sodium formate, oligomeric or polymeric secondary components (known as cuprenes), C₅ and C₆ components which originate from impurities in the acetylene used or from secondary reactions in the butynediol synthesis. When no pure butynediol obtainable, for example, by distillation from technical-grade 1,4-butynediol is used, the water content in the feed is 5-89% by weight, particularly preferably 20-70% by weight. The 1,4-butynediol content is generally from 10 to 90% by weight, preferably 20-80% by weight, particularly preferably 30-70% by weight. The propynol content is generally below 10% by weight, preferably below 5% by weight, particularly preferably below 3% by weight. Formaldehyde calculated as sum of formaldehyde itself, hydrate, acetal or hemiacetal has a content of below 5% by weight, preferably below 2% by weight, particularly preferably below 1% by weight. The methanol content is below 50% by weight, preferably below 5% by weight, particularly preferably below 1% by weight. The content of nonvolatile constituents is generally below 2% by weight, preferably below 1% by weight, particularly preferably below 0.1% by weight.

The vaporization of the 1,4-butynediol-comprising stream is generally carried out at pressures which correspond to at least the later hydrogenation pressure. However, it is also possible to choose a higher, for example up to 0.5 MPa higher, pressure in the vaporization of the 1,4-butynediol. Pressure drops which occur, for example as a result of pipes, valves, catalysts, heat exchangers, can be compensated by such an elevated pressure.

The vaporization of the 1,4-butynediol is carried out in the presence of hydrogen-comprising gas in apparatuses which are known per se for vaporization, for example in one or more falling film evaporators, thin film evaporators, helical tube evaporators, one-fluid or multifluid nozzles, tubes filled with inert materials in cocurrent or countercurrent with hydrogen, natural convection vaporizers, forced circulation vaporizers, kettle-type vaporizers or steam boilers. Here, the 1,4-butynediol-comprising gas stream can be heated further in order to achieve the desired reactor inlet temperature of the hydrogenation reactor.

As heat transfer medium for the vaporization, it is possible to use an appropriately preheated hydrogen-comprising gas stream which can comprise hydrogen together with helium, nitrogen and carbon dioxide and, if recycle gas from the hydrogenation is used, methane, ethane, propane, butane, methanol, dimethyl ether, ethanol, propanol, butanol, carbon monoxide, THF and water. These components are present in a proportion by mass of the gas stream which is generally below 50%, preferably below 40%, preferably below 30%. However, it is also possible to produce indirect heat exchange, for example by means of electric heating or heat transfer media such as steam or oil.

Components having boiling points higher than that of butynediol can be added to the 1,4-butynediol-comprising feed stream for the vaporization, for example when high boilers such as cuprenes and salts are present in the butynediol-comprising stream. These high-boiling components prevent solidification, e.g. of the salts, in the vaporizer. These high boilers can be, for example, alcohol-, ester-, ether-, urea-, urethane-, amide-comprising substances. Examples which may be mentioned are glycerol and oligomers thereof and Sokalan grades. The high boilers, which can be added in an amount of from 0.001 to 5% by weight based on butynediol, are then discharged together with the salts and cuprenes from the 1,4-butynediol and preferably burnt to produce energy.

The catalyst used in the process of the invention has at least one of the elements of groups 7 to 11 of the Periodic Table of the Elements as active component for the hydrogenation. These elements can be in the form of one or more metals or in the form of relatively low-valence compounds of these metals, for example as oxides, which are likewise hydrogenation-active. Among the hydrogenation-active elements of groups 7 to 11 of the Periodic Table of the Elements, the catalyst preferably has Mn, Re, Fe, Ru, Co, Rh, Ir, Ni, Pd, Pt, Cu and/or Au, particularly preferably Ru, Rh, Ir, Ni, Pd and/or Pt, as active components, very particularly preferably Pd as sole active component, from the groups of the Periodic Table of the Elements.

M. M. Telkmar et al., Journal of Molecular Catalysis A: Chemical (2002), 187(1), 81-93, and R. Chaudhari et al., Applied Catalysis (1987), 29(1), 141-59) disclose that catalysts based on palladium are preferably used in the liquid-phase hydrogenation when incomplete conversion of 1,4-butynediol in the hydrogenation is desired and 1,4-butenediol is the desired product. It is therefore surprising that palladium catalysts as used in the process of the invention in the gas phase lead directly to THF.

The metal content (active component) of the catalysts which can be used in the process of the invention is generally 0.001-100% by weight. In the case of catalysts which are generally produced by metal salt precipitation, the metal content is 1-100% by weight, preferably from 5-90% by weight, particularly preferably 10-80% by weight. In the case of catalysts which are produced by impregnation, the metal content is from 0.001 to 50% by weight, particularly preferably from 0.01 to 20% by weight, particularly preferably from 0.1 to 10% by weight.

The catalysts which are used in the process of the invention can additionally comprise at least one element or element compound selected from among the elements of groups 1 to 16 of the Periodic Table of the Elements and the lanthanides. These elements or element compounds can be comprised as a result of the method of production or else can be added deliberately, for example as promoter for the reaction and/or as support for the active component. The catalyst used in the process of the invention is preferably a supported catalyst.

The promoter content of the catalyst can be up to 25% by weight, preferably from 0.001 to 15% by weight, particularly preferably from 0.01 to 13% by weight. Mention may be made by way of example of alkali metal or alkaline earth metal components such as hydroxides, oxides, carbonates or salts of organic or inorganic acids, e.g. to vary the basic properties of the catalyst. Furthermore, sulfur, phosphorus, silicon and aluminum components can serve to modify catalysts in terms of their acid property. For example, sulfuric acid or phosphoric acid can be anchored on the catalyst. Particularly in the case of catalysts based on carbon, e.g. activated carbons, the acidic property of the support can also be adjusted by treating the carbon with, for example, hydrochloric, sulfuric, phosphoric or nitric acid. This can be carried out before or after impregnation with active component, preferably before.

As catalysts, it is possible to use precipitated, supported or Raney-type catalysts, the production of which is described, for example, in Ullmanns, Encyclopädie der technischen Chemie, 4th edition, 1977, volume 13, pages 558-665.

As support materials of the supported catalysts which are preferably used in the process of the invention, it is possible to use, for example, aluminum oxides, titanium oxides, zirconium dioxide, silicon dioxides, silicon carbide, sheet silicates, clay minerals, e.g. montmorillonites, silicates such as magnesium silicate or aluminum silicates, zeolites and also activated carbons. Preferred support materials are aluminum oxides, titanium dioxides, silicon dioxide, zirconium dioxide and activated carbons. Of course, it is also possible to use mixtures of various support materials as support for catalysts which can be employed in the process of the invention.

These supports can also be prefabricated monoliths, e.g. of ceramic, SiO₂, Al₂O₃, etc., or, for example, corrugated sheets which are later rolled together so that they give a cylindrical shape through which flow can occur or, for example, wire knitteds which can likewise be shaped.

Suitable catalysts for the hydrogenation according to the invention of 1,4-butynediol to THF are heterogeneous catalysts which are preferably used as shaped bodies. For the purposes of the present patent application, shaped bodies are, for example, crushed material, extrudates and pellets. These shaped bodies can also be hollow bodies, for example hollow cylinders, stars and trilobes, in order to increase the surface area. Catalysts in the form of crushed material, pellets and extrudates are preferably used in the process of the invention. These shaped bodies have diameters of 0.1-20 mm, preferably 1-10 mm, particularly preferably 1.5-7 mm. The length of the catalyst bodies is not critical but should generally be not less than the diameter. Preferred lengths of the shaped catalyst bodies are 1-50 mm.

The supported catalysts used according to the invention are produced by application of the active component or the combinations of active components, which can be applied together or in succession. Application can be effected by methods known per se, for example by impregnation, precipitation, sputtering. The active components are generally present as a thin layer on the support. In the case of application by sputtering, the content of active material based on the support can also be below 0.001% by weight. In this case, a content of active component of 0.00001-0.5% by weight is preferred.

Furthermore, Raney-type catalysts, for example Raney nickel, Raney copper, Raney cobalt, Raney nickel/molybdenum, Raney nickel/copper, Raney nickel/chromium, Raney nickel/chromium/iron, Raney nickel/palladium or rhenium sponge, are suitable for the process of the invention. Raney nickel/molybdenum catalysts can be produced, for example, by the process described in U.S. Pat. No. 4,153,578. However, these catalysts are also marketed, for example, by Degussa, 63403 Hanau, Germany. A Raney nickel-chromium-iron catalyst is marketed, for example, under the trade name Katalystor Typ 11 112 W® by Degussa.

The Raney catalysts are likewise preferably used as shaped bodies, e.g. tabletted or extruded, but it is likewise possible to treat alloy granules with, for example, sodium hydroxide in such a way that only an outer layer of the particle is leached to expose the active Raney layer. Such particles then have, for example, diameters in the range from 1 to 10 mm.

When precipitated or supported catalysts are used, these are preferably reduced at from 20 to 500° C. in a stream of hydrogen or hydrogen/inert gas before commencement of the reaction; this can be effected, for example, during heating of the reactor to the start temperature in the presence of hydrogen. The reduction temperature depends on the desired degree of reduction and the temperature necessary for the active component. Thus, Pd, which is present, for example, as PdO on a support requires temperatures of 20-100° C., while Co oxides require 200-300° C. for activation by means of hydrogen. This reduction can be carried out directly in the hydrogenation reactor. If the reduction is carried out in a separate reactor, the catalysts can be passivated on the surface at, for example, 30° C. by means of oxygen-comprising gas mixtures before removal from the reactor. The passivated catalysts can in this case be activated upstream of nitrogen/hydrogen at, for example, 180° C. in the hydrogenation reactor before use or else be used without activation.

The process of the invention can be carried out using one type of catalyst. However, it is also possible to use mixtures of a plurality of catalysts. These mixtures can be present as a pseudo-homogeneous mixture or as a structured bed in which individual reaction zones having a pseudohomogeneous catalyst bed are present. It is also possible to combine the methods, i.e., for example, to use one catalyst type at the beginning of the reaction and use a mixture further on.

In a preferred embodiment of the process of the invention, an acidic catalyst which has no hydrogenation properties but is capable of converting 1,4-butanediol into THF and water is used in addition to at least one of the above-described catalysts which has at least one of the elements of groups 7 to 11 of the Periodic Table of the Elements as active component for the hydrogenation. In this preferred embodiment of the process of the invention, aluminum oxides, titanium oxides, zirconium dioxides, silicon dioxides, sheet silicates, clay minerals, e.g. montmorillonites, silicates such as magnesium or aluminum silicates, zeolites and also acidified activated carbons can be used as acidic catalyst. To increase the acidity, it is possible to employ the usual methods, e.g. application of sulfuric acid or phosphoric acid. It is important here that no basic components such as amines or possibly volatile or entrained salts which can neutralize the acid sites get onto the acidic catalyst.

There are a number of possibilities for the industrial implementation of the hydrogenation. After vaporization of the 1,4-butynediol or the 1,4-butynediol-comprising stream, preferably by means of a stream of hydrogen and heating, the butynediol-comprising gas stream goes into the hydrogenation reactor. After the hydrogenation, the gas stream is cooled, product is largely separated off from hydrogen and the product stream is worked up further. The remaining hydrogen is partly discharged, and a part is recirculated, preferably as recycle gas.

The hydrogenation is carried out using a gas stream which comprises fresh hydrogen and, in addition to hydrogen, can further comprise, for example, helium, nitrogen and carbon dioxide in a proportion by mass of the gas stream which is generally below 5%, preferably below 1%, particularly preferably below 0.5%.

The space velocity over the catalyst in the hydrogenation according to the invention is generally from 0.01 to 3 kg of 1,4-butynediol (l of catalyst.h). Preference is given to space velocities over the catalyst of from 0.05 to 2, particularly preferably from 0.1 to 1, kg of 1,4-butynediol/l of catalyst.h.

The molar ratio of hydrogen consumed chemically by hydrogenation to 1,4-butynediol used is, for advantageous reaction, at least 2:1, preferably 2-4:1. After the reaction, excess hydrogen can be discharged. Preference is given to having a relatively high molar ratio of hydrogen to butynediol or reaction products thereof, for example 4-400:1, preferably 20-300:1, particularly preferably 40-200:1, during vaporization or during the reaction.

This is achieved by the preferred recycle gas mode in which at least part of the hydrogen or the hydrogen-comprising gas stream is circulated. The amount of hydrogen consumed chemically by hydrogenation and also by discharge is replaced. In a preferred embodiment, part of the recycle gas is discharged in order to remove inert compounds. The hydrogen which is circulated can also be utilized for vaporizing the 1,4-butynediol stream in the process of the invention.

The proportion of hydrogen discharged, calculated as proportion of hydrogen consumed chemically by hydrogenation, is from 100 to 0.1%. Preference is given to from 50 to 0.2%, particularly preferably from 20 to 0.3%. The lower the ratio, the less hydrogen is discharged and the more economical is the process. The discharge can be carried out either via offgas or else via the hydrogen dissolved in the cooled, liquid product stream. Discharge is carried out to remove inerts or secondary components in a targeted manner. Inerts can, for example, be introduced by the hydrogen, e.g. He, N₂, CO₂. Secondary components can be, for example, methane, ethane, propane, butane, methanol, dimethyl ether, ethanol, propanol, butanol and carbon monoxide which are formed in the reaction. Further components in the recycle gas stream can be desired reaction products such as THF and also water. It is advantageous for the inerts, products, water and secondary components to be present in the recycle gas in concentrations which are not too high, since they reduce the partial pressure of hydrogen. They should be present in the recycle gas in a proportion of less than 50%. A special case is carbon monoxide, possibly also carbon dioxide, since these can reduce the activity of the active components. The proportion of carbon monoxide and/or carbon dioxide in the recycle gas should therefore ideally be below 10%, preferably below 5%, particularly preferably below 1%.

A considerable advantage of the process of the invention is that THF comprised in the recycle gas decomposes only insignificantly if at all. It has surprisingly been found that THF remains unchanged to an extent of at least 99%, even after a second pass through the catalyst. Although complete condensation of the product is desirable, the minimum condensation temperature required for condensing the product stream is not critical because of the above-described stability of the THF, as a result of which energy can be saved. The condensation temperature is generally from −78° C. to 70° C., preferably from −15° C. to 40° C., particularly preferably from −10° C. to 30° C.

The temperature conditions in the hydrogenation reactor are preferably such that the temperature increases along at least ¼ of the length of the catalyst bed, independently of the type of reactor used. If cooling were to be brought about along the catalyst bed, especially in the first zone of the catalyst, there would be the risk of condensation which would then lead to deactivation of the catalyst as a result of carbon deposits. The temperature increase along at least the first quarter of the catalyst bed is 1-100° C., preferably from 2 to 80° C., particularly preferably from 5 to 60° C. After the maximum temperature has been reached, the reactor temperature can, depending on the type of reactor (e.g. shell-and-tube reactor), decrease again. Here, the entry temperature is the temperature of the gas stream with which the stream impinges on the catalyst.

After leaving the catalyst bed or the hydrogenation reactor, the gas stream (hydrogenation output) is cooled in one or more stages to such an extent that a liquid phase, viz. the product mixture, is formed. This preferably occurs at the same pressure level as the reaction itself. Cooling can be effected by means of air and water coolers, refrigeration plants or other industrial auxiliaries.

As reactor or as types of reactor for the hydrogenation according to the invention in the gas phase, it is possible to use, for example, shell-and-tube reactors, shaft reactors or fluidized-bed reactors. A special case would be the microreactor which is particularly advantageous when the heat of reaction is to be removed very efficiently in order to keep the temperature of the reaction as constant as possible. The reactors or types of reactor used can also be employed as a combination. The process of the invention is preferably carried out continuously.

Before the work-up, the liquid product mixture is generally depressurized to a lower pressure level, if the hydrogenation reaction was carried out under superatmospheric pressure, for example to 0.1 to 0.5 MPa absolute. This results in liberation of dissolved hydrogen-comprising gas which is either discharged or recirculated.

The liquid product mixture can subsequently be fractionated by known methods, preferably by distillation. In the case of industrial processes, fractionation is preferably carried out continuously in a plurality of columns. A work-up can, for example, be as follows: the liquid hydrogenation output goes into a first column (a) in which all the THF together with water, preferably only part of the water corresponding to the THF/water azeotrope at the pressure set, and other low boilers are separated off from the relatively high-boiling components at pressures of from 0.05 to 0.3 MPa absolute. The THF-comprising stream is fractionated further in a second column (b), preferably at pressures above those in the first column, for example at from 0.15 to 1.5 MPa absolute. The THF/water low-boiling azeotrope obtained here can be recirculated in its entirety or only partly to the first column. Low boilers present, e.g. methanol, can be entirely or partly discharged at the top of the column. Since these low boilers, for example methanol, still comprise THF, this mixture can, depending on the amount, be fractionated further in one or more separate column(s). The high-boiling product from the column (b), viz. THF, can be saleable as such, and if purities above 99.9% are wanted can be subjected to final purification in a further column. The high-boiling product from the column (a), which usually comprises predominantly water, can, for example, be passed to disposal (water treatment plant or incineration).

One variant of this work-up is, especially when methanol contents of >0.2% are present in the hydrogenation output, to accumulate the methanol in the column combination (a)+(b) by recirculation of a THF/methanol-comprising low boiler stream from (b) to (a) until no more water goes over together with the methanol/THF azeotrope from column (a) to the column (b). In this way, all water together with excess methanol is discharged via the bottom of the column (a).

If products of value, e.g. 1,4-butanediol and/or gamma-butyrolactone, are still comprised, these can either be isolated in pure form or, after removal of water and other undesirable components such as propanol and butanol, be recirculated to the reaction. A preferred variant is to treat the high boiler stream from column (a), if it still comprises 1,4-butanediol, by means of an acidic catalyst so as to cyclize the butanediol to THF. This can be carried out using the high-boiling stream from the column (a) directly or after separating off components so that the butanediol is present in concentrated form. Catalysts for this cyclization can be homogeneously dissolved or be present heterogeneously as suspension or fixed bed. The cyclization can in the case of fixed-bed catalysts be carried out in the gas or liquid phase, otherwise in the liquid phase. The temperatures here are in the range from 80 to 300° C., preferably from 90 to 250° C. As catalysts, mention may be made of inorganic acids such as sulfuric acid, phosphoric acid or heteropolyacids such as tungstophosphoric acid, organic acids such as sulfonic acids, acidic ion-exchange resins and acidic, inorganic solids such as zeolites, metal oxides such as SiO₂ or Al₂O₃, mixed metal oxides such as montmorillonites.

The quality of the THF is guided by commercial standards. Thus, the THF satisfies the usually required GC purities and the color numbers and other characteristic properties. The invention is illustrated by the following examples.

### EXAMPLES

The analysis of the product was carried out by gas chromatography. The product compositions indicated are all calculated on a water-free basis. The 1,4-butynediol used was prepared by reaction of acetylene with formaldehyde over Cu/Bi catalysts of the Reppe type. The technical-grade 1,4-butynediol used comprised 50% by weight of butynediol, 47% by weight of water, 1% by weight of formaldehyde, about 1% of propynol and small amounts of further components such as cuprenes, Cu compounds, salts such as sodium formate. The pure 1,4-butynediol was present as 40% strength aqueous solution. The conversion indicated is that of the 1,4-butynediol used. The selectivity figures take into account 1,4-butynediol or intermediates still to be hydrogenated. Here, 1,4-butenediol, 4-hydroxybutyraldehyde and acetals derived therefrom, gamma-butyrolactone (GBL) or butanediol (BDO) were taken into account as intermediates still to be hydrogenated or reacted to form THF.

### Example 1

In the example, the hydrogenation was carried out using pure 1,4-butynediol as 40% strength aqueous solution under atmospheric pressure. This 1,4-butynediol solution was pumped continuously into an externally heated reactor tube (diameter 2.7 cm). The tube was provided with glass rings (30 ml) in the upper region. This zone served as vaporizer section in which butynediol/water and hydrogen (300 liter/h) were heated to the hydrogenation temperature indicated in table 1 and fed in gaseous form to a second zone (hydrogenation zone) in the reactor tube in which the catalyst indicated in table 1 (20 ml) was located. Downstream of the reactor tube, the gaseous reactor output was cooled to about 20° C. and condensed product was collected. The offgas was passed through a cold trap at −78° C. and further product was condensed out. The two condensates were combined for the purpose of analysis. The catalyst was activated in a stream of hydrogen before the reaction. The result is shown in table 1.

TABLE 1

| | Space velocity | | | | | | |
|---|---|---|---|---|---|---|---|
| | over the catalyst | | THF¹ | BDO² | GBL³ | BYD⁴ | |
| | (kg of butynediol/ | Temperature | (% by | (% by | (% by | conversion | Selectivity |
| Example/catalyst | liter of cat · h) | (° C.) | weight) | weight) | weight) | (%) | (%) |
| 1 | 0.5 | 225 | 47.3 | 16.3 | 1.6 | 99.8 | 65.5 |
| 5%Pd/C | | | | | | | |

¹THF = tetrahydrofuran
²BDO = 1,4-butanediol
³GBL = gamma-butyrolactone
⁴BYD = 1,4-butynediol

### Examples 2 to 5

In these examples, aqueous pure 1,4-butynediol or technical-grade 1,4-butynediol in example 5 (denoted by *) was vaporized in a stream of hydrogen under superatmospheric pressure in a vaporizer which comprised metal filling rings and was externally heated to 240° C. by means of oil and hydrogenated in a reactor tube filled with catalyst (100 ml unless indicated otherwise in table 2) or catalysts. The reactor tube was configured as a double-walled tube which was heated or cooled externally by means of oil. The reaction output was cooled and the condensed product was depressurized via a valve to atmospheric pressure, while the gas phase was recirculated via a recycle gas fan to the vaporizer. A small part of the gas was discharged as offgas. Hydrogen was introduced by means of a fresh gas supply into the reaction in the amount corresponding to the hydrogen consumed by reaction and offgas. The reaction pressure was kept constant in this way. The experimental results are shown in table 2. The conversion of butynediol was in all cases quantitative. To calculate the selectivity, products such as 1,4-butenediol and 4-hydroxybutanal and acetals thereof, gamma-butyrolactone (GBL) and butanediol (BDO) were included in the calculation as compounds still to be hydrogenated or reacted to form THF.

TABLE 2

| | Space velocity | | | | | | |
|---|---|---|---|---|---|---|---|
| | over the catalyst | | | THF¹ | BDO² | GBL³ | |
| | (kg of butynediol/ | Temperature | Pressure | (% by | (% by | (% by | Selectivity |
| Example/catalyst | liter of cat × h) | (° C.) | (MPa) | weight) | weight) | weight) | (%) |
| 2 | 0.2 | 220-230 | 0.5 | 75 | 15 | 1 | 93 |
| 0.5%Pd/C | | | | | | | |
| 3 | 0.2 | 220-230 | 0.9 | 79 | 11 | 1 | 92 |
| 0.5%Pd/ZrO₂ | | | | | | | |
| 4 | 0.3 | 225-240 | 0.9 | 82 | 10 | 0.2 | 94 |
| 0.5%Pd/C (20 ml) + | | | | | | | |
| 0.5%Pd/ZrO₂ (40 ml) | | | | | | | |
| 5* | 0.1 | 220-225 | 0.5 | 80 | 0 | 3 | 85 |
| 0.5%Pd/Zr0₂ (90 ml) + | | | | | | | |
| 20 ml Al₂O₃ | | | | | | | |

¹THF = tetrahydrofuran
²BDO = 1,4-butanediol
³GBL = gamma-butyrolactone

Reworking of the examples of DE-A 2029557

### Example 6 of DE-A 2029557

As described in example 6 of DE-A 2029557, 140 g of 1,4-butynediol in 360 g of water were hydrogenated in the liquid phase at 10.3 MPa and 275° C. over a catalyst comprising 5% of palladium on activated aluminum oxide. Only decomposition products and water were found in the hydrogenation output. THF was not found.

### Examples 2, 3 and 4 of DE-A 2029557

Butynediol was hydrogenated as described in examples 2, 3 and 4 of DE-A 2029557. The hydrogenation output from the reworking of example 2 using Pd on activated aluminum oxide spheres as catalyst comprised mainly n-butanol and water and <3% by weight of THF. In the case of Ni- and Ru-comprising catalysts as per examples 3 and 4, only water was found in the output. In addition to a little (<1%) of THF, mainly hydrocarbons from methane to butane were detected in the offgas.

### Example to compare with DE-A 2029557

Using a method analogous to example 6 of DE-A 2029557, a 40% strength by weight aqueous butynediol solution was hydrogenated in the liquid phase at 10.3 MPa and 225° C. over a catalyst comprising 5% of palladium on activated aluminum oxide (5% Pd/Al₂O₃). Apart from polymeric residues, many components were present in the liquid hydrogenation output. About 14% by weight of n-propanol, 10% by weight of n-butanol and 20% by weight of THF were found. The total proportion of organic components in the hydrogenation output corresponded to 10% of the original amount of butynediol used. The selectivity to THF was only 2%.

### Example 6

According to the invention, a 40% strength by weight aqueous butynediol solution was hydrogenated in the gas phase at an entry temperature of 225° C. and 0.9 MPa over a catalyst comprising 5% of palladium on activated aluminum oxide (5% Pd/Al₂O₃). 75% by weight of THF, 20% by weight of n-butanol and less than 1% by weight of each of gamma-butyrolactone, 1,4-butanediol and n-propanol were found in the hydrogenation output. The selectivity to THF was 75%, since the conversion of the butynediol was complete.

1. A process for preparing tetrahydrofuran by hydrogenation of 1,4-butynediol, wherein 1,4-butynediol is vaporized in a hydrogen-comprising gas stream and is hydrogenated in gaseous form over at least one catalyst, comprising at least one of the elements from groups 7 to 11 of the Periodic Table of the Elements.

2. The process according to claim 1, wherein the temperature at which the 1,4-butynediol is vaporized and the hydrogenation temperature are each from 160 to 300° C.

3. The process according to either claim 1 or 2, wherein the pressure at which 1,4-butynediol is vaporized corresponds to at least the pressure of the subsequent hydrogenation.

4. The process according to any of claims 1 to 3, wherein the hydrogenation is carried out at a pressure of from 0.5 to 10 MPa.

5. The process according to any of claims 1 to 4, wherein the temperature at which 1,4-butynediol is hydrogenated is from 160 to 300° C.

6. The process according to any of claims 1 to 5, wherein the catalyst comprises at least one of the elements selected from among Mn, Re, Fe, Ru, Co, Rh, Ir, Ni, Pd, Pt, Cu and Au.

7. The process according to any of claims 1 to 6, wherein the catalyst comprises Pd as active metal.

8. The process according to any of claims 1 to 7, wherein two catalysts comprising elements of groups 7 to 11 of the Periodic Table of the Elements are used, with one of the catalysts comprising Pd as active metal.

9. The process according to any of claims 1 to 8, wherein the catalyst(s) comprises a support selected from among the oxides of aluminum, of titanium, zirconium oxide, silicon oxide, clay minerals, silicates, zeolites and activated carbon.

10. The process according to any of claims 1 to 9, wherein an acidic catalyst without hydrogenation properties is additionally used.

11. The process according to any of claims 1 to 10, wherein the molar ratio of hydrogen to 1,4-butynediol fed in is at least 2 during the hydrogenation.

12. The process according to any of claims 1 to 11, wherein the hydrogenation is carried out using the recycle gas mode.
